(19) United States
(12) Patent Application Publication
(10) Pub. No.: US 2024/0415541 A1
(43) Pub. Date: Dec. 19, 2024

(54) UTERINE TAMPONADE DEVICE

(71) Applicant: COOPERSURGICAL, INC., Trumball, CT (US)

(72) Inventors: Jeffry S. Melsheimer, Springville, IN (US); Raymond G. Amos, Spencer, IN (US)

(21) Appl. No.: 18/704,996

(22) PCT Filed: Oct. 28, 2022
(86) PCT No.: PCT/US2022/048264

§ 371 Date: Apr. 26, 2024

Related U.S. Application Data
(60) Provisional application No. 63/276,098, filed on Nov. 5, 2021.

(51) Int. Cl. A61B 17/42; A61M 25/10

(52) U.S. Cl. CPC A61B 17/42; A61M 25/10; A61B 2017/4216; A61M 2025/1068

(57) ABSTRACT

A tamponade device may include: a catheter, where a catheter lumen extends from a proximal end of the catheter to a distal end of the catheter; an inflatable balloon located at the distal end of the catheter; and a constraint at least partially surrounding the inflatable balloon. In an expanded configuration, the constraint may contact an outer perimeter surface of the balloon such that the constraint limits a radial expansion of the balloon when the balloon is inflated.

### RELATED APPLICATION(S)

This application claims the benefit of U.S. Provisional Application No. 63/276,098, titled “UTERINE TAMPONADE DEVICE,” and filed Nov. 5, 2021, which is hereby incorporated by reference in its entirety.

### TECHNICAL FIELD

The present disclosure relates generally to devices and methods for controlling uterine bleeding, and more specifically, to devices for use with a uterine tamponade assembly having an inflatable balloon.

### BACKGROUND

Uterine bleeding is a clinical condition attributable to a variety of causes, including postpartum hemorrhages (PPH) following vaginal and/or cesarean childbirth. Postpartum hemorrhage or excessive blood loss after birth is commonly caused by uterine atony whereby the uterus fails to contract normally after the delivery of a baby, leading to continuous bleeding. If left untreated, PPH may cause serious complications or even death.

There are a variety of techniques used for treating and managing PPH, including the administration of muscle contracting drugs or agents alone or in combination with other mechanical or surgical techniques. One such technique includes inserting a tamponade apparatus, such as a balloon catheter into the uterus, wherein the balloon is inflated to a sufficient pressure and volume until it conforms generally to the contour of the uterine cavity. The application of pressure to the interior uterine wall provides a tamponade effect until bleeding is controlled or stopped. One example of a uterine tamponade balloon catheter is the Bakri® balloon by Cook Medical (Bloomington, Indiana). The effectiveness of the Bakri® balloon may be partially attributable to maintaining the balloon in a proper position within the uterine cavity, and more specifically, in the lower uterine segment.

While the Bakri® balloon is currently used with success, the present application describes features and aspects that may improve or otherwise build upon this technology.

### BRIEF DESCRIPTION OF THE DRAWINGS

Certain features, aspects, and advantages of the disclosed embodiments are shown in the drawings accompanying this description. The drawings are briefly described below.

FIG. 1 is an illustration showing a tamponade device having an expandable balloon in accordance with certain aspects of the present disclosure.

FIG. 2 is an illustration showing the tamponade device having the expandable balloon covered by a constraint in a non-inflated state in accordance with certain aspects of the present disclosure.

FIG. 3 is an illustration showing the constraint from FIG. 2 in a delivery (or non-expanded) configuration in accordance with certain aspects of the present disclosure.

FIG. 4 is an illustration showing the constraint of FIG. 3 in an expanded configuration in accordance with certain aspects of the present disclosure.

FIG. 5 is an illustration showing the tamponade device in an expanded configuration, where the balloon is inflated and limited in the radial direction by the constraint in accordance with certain aspects of the present disclosure.

FIG. 6 is an illustration depicting another embodiment of a constraint for the tamponade device, where the constraint includes tubes having openings for delivery of a fluid to the uterus wall in accordance with certain aspects of the present disclosure.

FIG. 7 is an illustration depicting the constraint of FIG. 6 in an expanded state in accordance with certain aspects of the present disclosure.

FIG. 8 is an illustration depicting another embodiment of a constraint for the tamponade device, where the constraint includes a net at least partially formed by tubes in accordance with certain aspects of the present disclosure.

FIG. 9 is an illustration depicting an embodiment of a tamponade device having an inflatable balloon, where the inflatable balloon includes a helical portion in accordance with certain aspects of the present disclosure.

FIG. 10 is an illustration showing the tamponade device of FIG. 9, where the balloon is inflated in accordance with certain aspects of the present disclosure.

FIG. 11 is a section view about section 11-11 of FIG. 10 such that FIG. 11 depicts certain internal features of the tamponade device in accordance with certain aspects of the present disclosure.

### DETAILED DESCRIPTION

All publications, patent applications, patents, and other references mentioned herein are incorporated by reference in their entirety. Unless otherwise defined, all technical and scientific terms used herein have the same meaning as commonly understood by one of ordinary skill in the art to which this disclosure pertains. In the case of conflict, the present document and definitions will control.

The terms “proximal” and “distal” and derivatives thereof will be understood in the frame of reference of a medical physician using the device. Thus, proximal refers to locations closer to the physician and distal refers to locations farther away from the physician (e.g., deeper in the patient's vasculature or body cavity).

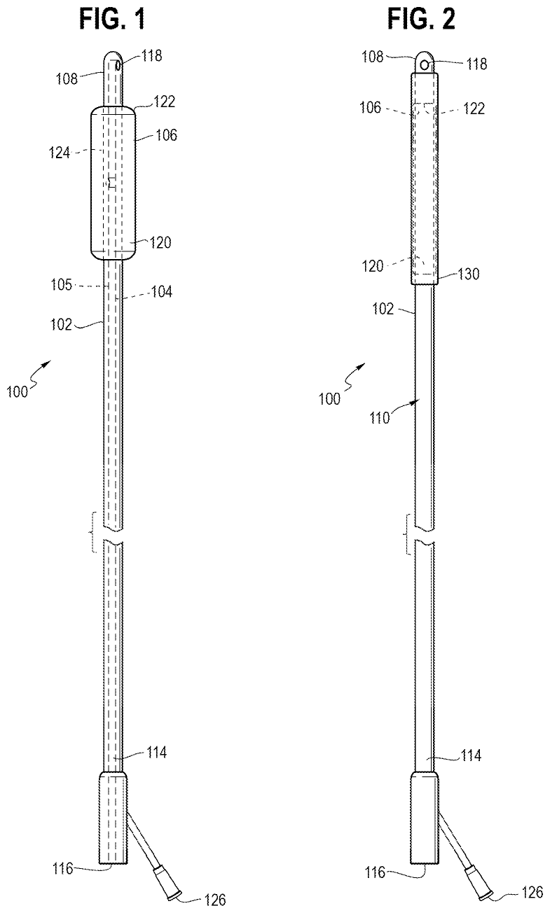

FIG. 1 illustrates a system for treating postpartum hemorrhage, which generally includes a tamponade device 100. As shown, the tamponade device 100 includes an elongate or elongated tube or other elongated supporting device, which in this instance is depicted as a flexible catheter 102 having a lumen 104 extending therethrough. An expandable device, such as an inflatable balloon 106, may be coupled to the distal end 108 of the catheter 102. For example, the balloon 106 may generally extend radially away from an outer perimeter surface 110 of the catheter 102 when the balloon 106 is in an inflated state.

With regards to the catheter 102, the catheter 102 may be in the form of a traditional flexible silicone catheter commonly used in treating postpartum hemorrhage for delivering an inflatable balloon to the uterus through the cervix. The depicted catheter 102 has an elongate form and includes a proximal end 114 and a distal end 108. The catheter 102 may include one or more lumens extending from the proximal end 114 toward the distal end 108. For example, the catheter 102 may include a drainage lumen 104 and an inflation lumen 105 (perhaps in addition to other lumens).

The catheter 102 may include a drainage lumen 104 extending from the proximal end 114 toward the distal end 108, with the drainage lumen 104 having a drainage access opening 116 located at the proximal end 114 of the catheter 102. The drainage lumen 104 extends from the drainage access opening 116 in a distal direction toward one or more drainage ports 118, with the drainage lumen 104 being in fluid communication with the drainage ports 118 such that fluid outside of the catheter 102 can flow through the ports 118 into drainage lumen 104 and can flow through the drainage lumen 104 toward and out of the drainage access opening 116 of the drainage lumen 104. It will be appreciated that the drainage lumen 104, being in fluid communication with the exterior of the catheter 102 to receive fluid into drainage lumen, is thereby arranged within the catheter 102 to access the exterior of the catheter 102.

While the drainage ports 118 are depicted as being located distally relative to the balloon 106, it is contemplated additional drainage port(s), or all drainage port(s) of the device, may be located proximally relative to the balloon. For example, drainage ports may be located along the catheter 102 in a manner similar or identical to the embodiments described in U.S. Pat. No. 10,813,668, titled “POSTPARTUM HEMORRHAGE BALLOON SYSTEM,” which is hereby incorporated by reference in its entirety. Notably, the present disclosure also covers embodiments without drainage ports altogether, particularly where “ravines” provided by the constraint (discussed below) allow drainage to occur wholly separately from a catheter lumen.

With regard to the balloon 106, the balloon 106 may be in the form of a traditional inflatable balloon for use in internal uterine compression for treating PPH, such as the Cook Bakri® Balloon. The balloon 106 may be inflatable from a radially compressed delivery configuration tight to the shaft (not shown) to a radially expanded and inflated configuration, in which the balloon 106 will exert an outward force on the internal surface of the uterus. The balloon 106 is preferably delivered to the uterus in a relatively small delivery configuration through the cervix, and then radially expanded and deployed into the inflated configuration once delivered within the uterus.

The balloon 106 may be attached to the catheter 102 in any suitable manner. For example, a proximal end 120 of the balloon 106 may be attached to the catheter 102, and the distal end 122 of the balloon may also be attached to the catheter 102. The balloon 106 may define an internal cavity 124 that is fluidly isolated from the exterior of the balloon 106, such that introduction of fluid (gas or liquid) into the cavity 124 will cause the balloon 106 to expand. To allow a medical professional to expand the balloon 106, the catheter 102 may have an inflation port or inlet 126 that is in fluid communication with the cavity 124 of the balloon 106, where the catheter 102 includes an inflation lumen 105 that extends from the proximal end 114 of the catheter to the inflation inlet 126, and such that there is fluid communication between the cavity 124 and the inflation lumen 105. The balloon 106 may be inflated or deflated by controlling the amount of inflation fluid that is introduced or removed from within the cavity 124 via the inflation lumen 105.

FIG. 2 shows the tamponade device 100 having a constraint 130 that at least partially surrounds the balloon 106 (where the balloon is in a deflated state in this image). As described in more detail below, the constraint 130 may generally limit the expansion of a balloon in the radial direction, thus preventing over-expansion and ensuring the extreme radial dimensions of the device are within an appropriate range. In addition (also discussed below in more detail), the constraint 130 may provide an inflated balloon with particular surface characteristics, such as a topography having “ravines” or channels for blood flow. In the following embodiments, the description describes the constraint as including a net. However, without limitation, the constraint may additionally or alternatively include a sleeve, band, ring, cuff, coil, pocket, or any other suitable device for constraining and/or imparting surface features onto an inflated balloon.

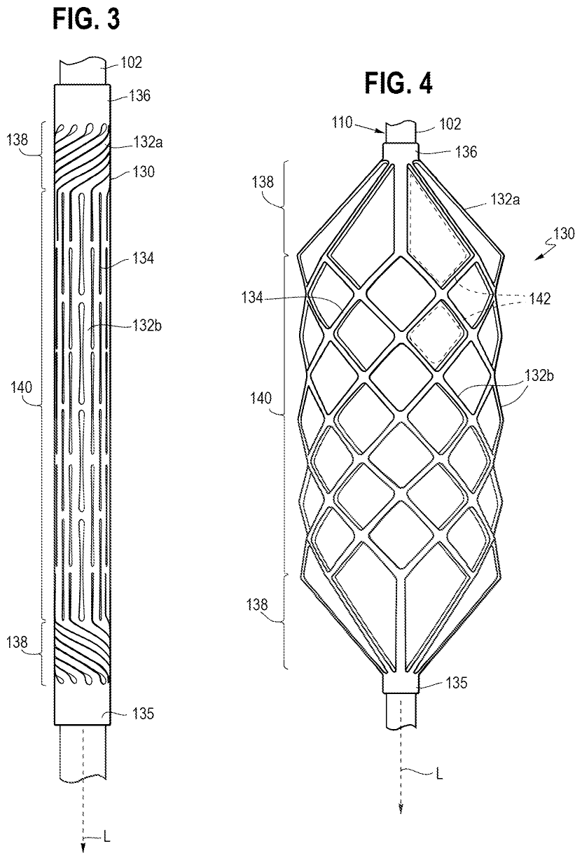

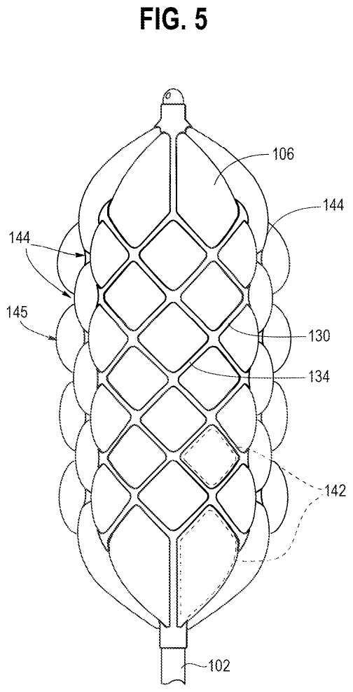

FIG. 3 and FIG. 4 show an embodiment of the constraint 130 with a net 134. FIG. 3 depicts the constraint 130 in a delivery configuration, and FIG. 4 depicts the constraint 130 in an expanded configuration. The transition between the delivery configuration of FIG. 3 and the expanded configuration of FIG. 4 may be caused by balloon inflation, which may force the net 134 radially outward. When removal of the device is desired and the balloon is deflated, the net 134 may have resilience such that it retains its FIG. 3 configuration, but this is not required and is not the case in all embodiments.

Referring to FIGS. 3-4, the constraint 130 includes a plurality of segments 132 that together form a net 134. Openings 142 are surrounded by/defined by the segments 132. The size of the segments 132 may be chosen to impart a particular shape and size of the openings 142. For example, when it is desirable for an inflated balloon to “bulge” through the openings (as shown in FIG. 5), the openings 142 may be larger than when such a feature is not desirable. Smaller openings 142, or embodiments without openings at all, may be desirable when the outer diameter of the expandable device must be relatively uniform and precisely controllable, where larger openings 142 may be advantageous when the above-mentioned bulging effect forms desirable ravines/channels for fluid flow (discussed in more detail below). Any suitable degree of bulging is contemplated. For example, in instances where the openings are in the form of a quadrilateral (e.g., diamond shaped), the distance an apex may extend beyond the openings may be about ½ to about ¼ the length of a side of the quadrilateral.

While the net 134 and its segments 132 may be formed in any suitable manner (e.g., weaving or knitting), the present embodiment's segments 132 are formed from the outer wall of a segment of flexible polymer tubing. For example, the proximal end 135 and the distal end 136 of the constraint may include uncut portions of the polymer tubing such that the polymer tubing retains its original form. The net 134 may include many precisely-cut features, for example using a laser cutter. These laser-cut features may be completely or only partially cut through the tubing. Any suitable tubing may be used (e.g., in one example, the original polymer tubing has an outer diameter of approximately 0.5 inches and a wall thickness of approximately 0.064 inches).

At least a portion of the net 134 may be slidable along the catheter 102 in the longitudinal direction. For example, as shown in FIG. 4, the proximal end 135 of the constraint 130 has moved distally relative to its position in FIG. 3 and the distal end 136 of the constraint has moved proximally relative to its position in FIG. 3. This reduction in size in the longitudinal direction may translate to a reorientation of certain net segments 132 such that the net 134 expands outward radially, thereby increasing its outer diameter. In some embodiments, the proximal end 135 may be fixed relative to the catheter 102 while the distal end 136 is slidable. In other embodiments, the distal end 136 may be fixed relative to the catheter 102 while the proximal end 135 is slidable. In other embodiments (including the depicted embodiment), both the proximal end 135 and the distal end 136 may be slidable such that they slide towards each other along the catheter 102 when the constraint expands. In yet other embodiments, it is contemplated that the distal end 136 and the proximal end 135 may remain substantially fixed in their locations along the catheter, and the net 134 may expand due to displacement elsewhere (e.g., the segments 132 of the net 134 may stretch or otherwise change in dimension, may twist or otherwise change their angle of orientation relative to the longitudinal direction, etc.).

Referring to the expanded configuration in FIG. 4, the segments 132a of two angled profile portions 138 are oriented such that they angle radially away from the longitudinal direction L and extend to a central portion 140 of the net 134, which is spaced radially outward from an outer perimeter surface 110 of the catheter 102. The central portion 140 of the net 134 may be substantially cylindrical, as shown, which may be advantageous for providing an approximately uniform, controllable outer diameter of the balloon when inflated.

Differentiation between the segments of the central portion 140 and those forming the angled profiles 138 may occur during the laser cutting process (or other cutting process). For example, referring back to FIG. 3, the segments 132a of the angled profile portion 138 are cut in a different pattern than the segments 132b of the central portion 140. Specifically, the segments 132a of the angled profile portion 138 are formed in a spiral pattern such that they are forced to “twist” into a straightened orientation as the net 134 expands radially outward until the segments 132 straighten into their configuration shown in FIG. 4. Each of these segments 132a moves in substantially the same direction as the net 134 expands. By contrast, the segments 132b of the central portion 140 of the net 134 move in alternating directions during expansion, thereby forming a diamond pattern extending around the outer periphery of a cylindrical shape.

While this particular net and segment structure of the net 134 of FIGS. 3-4 is described in detail above, it will be appreciated that other net and segment structures may be used to arrive at a similar result.

FIG. 5 shows the constraint 130 from FIGS. 3-4 in an expanded state along with the inflated balloon 106 from FIG. 1. As shown, the constraint 130 contacts the outer surface of the balloon 106 such that the constraint 130 limits the outer perimeter dimensions of the balloon 106 such that the balloon cannot expand into an overinflated state. Further, since the balloon 106 includes certain portions (bulges 145) that extend outwards through the openings 142 of the net 134, the outer surface of the balloon 106 has an irregular shape creating a heavily textured topography. As a result, multiple ravines 144 provide additional flow paths for blood to be drained from anywhere in the uterus. For example, assuming apex portions 146 of the balloon 106 press against the walls of the uterus, (where “apex portions” are defined as the point being the most radially-outward relative to the catheter 102 in a particular vicinity), space will be retained between the uterus wall and the net 134, thereby providing channels such that blood can bypass the balloon 106 as it flows through the uterus. In certain exemplary embodiments, the balloon may be formed of an elastomeric material (e.g., latex, silicone, polyurethane, silicone/polyurethane co-polymers, etc.), which may be advantageous for preventing the balloon 106 from folding over itself in an attempt to bulge through the openings.

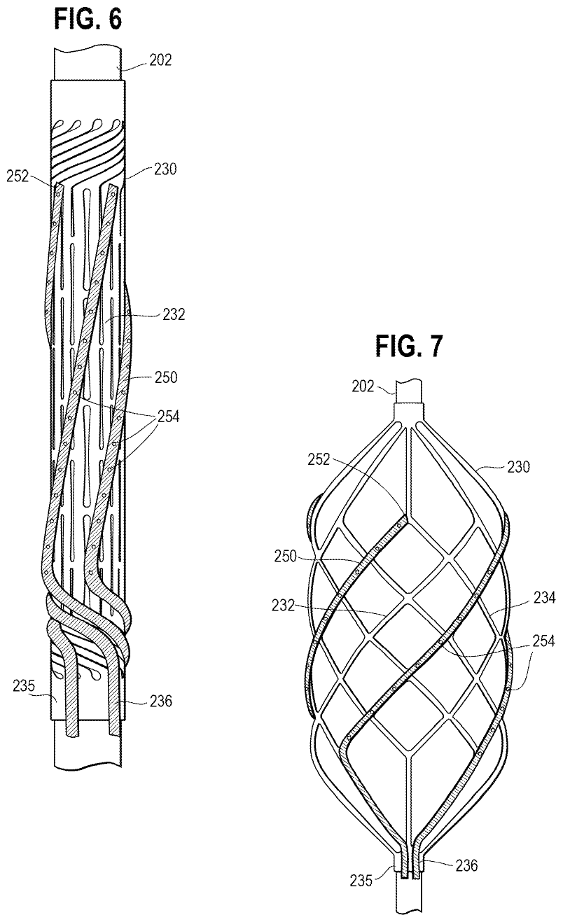

FIG. 6-7 show another embodiment of a constraint 230 in a delivery configuration and an expanded configuration, respectively. The constraint 230 may include any or all of the features and advantages discussed above. In addition, the constraint 230 includes a set of tubes 250 that, when in the expanded state of FIG. 7, extend along the outer diameter of the net 234. While any suitable tubes are contemplated, the tubes 250 of the depicted embodiment may have an outer diameter of about 0.125″ and 0.2965″ with a laser-drilled lumen therein, which may be suitable for suction/vacuum.

The tubes 250 are shown as being generally radially outside the segments 232 of the net 234, but this is not required. In other implementations, the tubes 250 may be woven through the net 234 or otherwise alternate outside/inside certain segments 232, or the tubes 250 may be generally on the inside of the net 234. Further, the tubes 250 may be secured to the segments 232 in any suitable manner, such as via an adhesive, sewing or tying, the use of a mechanical clamp, etc. For example, the tubes 250 may be rigidly fixed to the segments 232 only at the distal ends 252 of the tubes 250 and at the proximal end 235 of the constraint 230 (e.g., at point 236), but additional and/or alternative points of securement are also contemplated. The tubes 250 are shown as terminating at distal ends 252 within the net 234, but alternatively the tubes 250 may extend farther distally such that they extend to the extreme distal end of the constraint 230 and perhaps beyond.

The tubes 250 may extend proximally beyond the constraint 230, either outside or inside the catheter 202, such that the lumens extending through the tubes 250 are accessible on a proximal end of the tamponade device. Alternatively, to accomplish this same result, the tubes 250 may be coupled to a dedicated lumen within the catheter 202. Thus, the tubes 250 may be used to deliver various fluids to the uterus wall (e.g., where they remain are located within the patient for fluid introduction and drainage, for example). For example, the tubes 250 may deliver drugs to promote blood coagulation to stop postpartum hemorrhage, drugs to promote contraction of the uterus, drugs to promote shrinkage of the blood vessels in the uterine wall in turn stopping postpartum hemorrhage, etc. When the tubes 250 include holes or openings 254, these holes or openings 254 may be sized to optimize flow of a particular fluid and for appropriate delivery to the uterus wall.

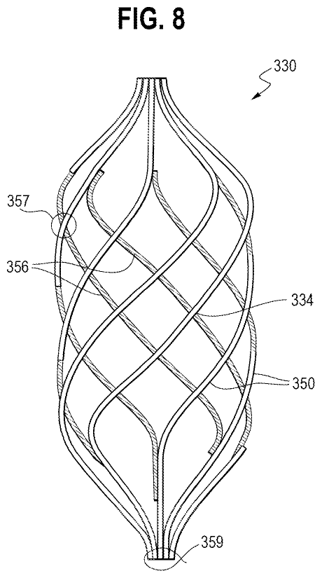

FIG. 8 shows another embodiment of a constraint 330. Like the embodiment shown in FIGS. 6-7, this embodiment includes a set of tubes 350, which may be configured for fluid delivery as discussed above. A distinction between this embodiment and the embodiment of FIGS. 6-7 is that the net 334 of the constraint 330 shown in FIG. 8 is primarily formed by the tubes 350 themselves. That is, the above-described segments cut from a polymer tube are not included in this embodiment, nor are they necessary, since the tubes 350 themselves form a net structure capable of constraining a tamponade balloon. Optionally, the tubes 350 may combine at a manifold 359, which may extend to the proximal end of the device and/or fluidly communicate with a lumen of an associated catheter.

To enhance the strength and durability of the net 334, the tubes 350 may extend in a helical pattern around the balloon and be substantially parallel to each other, and they may be optionally secured to one another via a plurality of elongated polymer elements 356 (e.g., polymer strips, tubes, or the like). These polymer elements 356 may angle relative to the tubes 350 within the net 334, such as via extending in an opposite-direction helical pattern, such that they form a criss-crossing pattern with the tubes 350. While not required, it is contemplated that the polymer elements 356 may be woven with the tubes such that they alternate radially outside and inside. At points of engagement, depicted as points 357, the tubes 350 may optionally be fixed relative to the polymer elements 356 to enhance the strength and durability of the net 334 during use. Without limitation, this securement may be obtained via an adhesive, sewing or tying, a mechanical clamp, weaving or knitting, etc.

In a similar embodiment, the polymer elements 356 shown in FIG. 8 may be substituted for another set of tubes (not shown). In other words, the number of tubes 350 shown in FIG. 8 may be approximately doubled, and the additional tubes may parallel to one another but angled relative to the tubes 350 already existing in FIG. 8. These additional tubes may extend about where the polymer elements 356 are located in FIG. 8, for example. Any other suitable embodiment involving the tubes 350 may also be included.

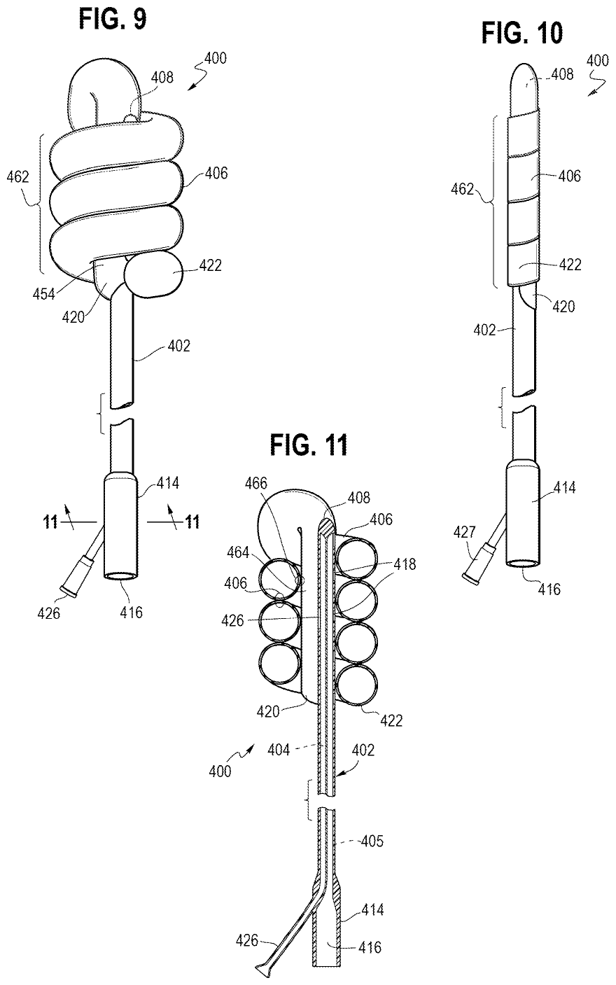

FIGS. 9-11 illustrate a tamponade device 400 having a balloon 406, which is similar in function to the balloon 106 shown in FIG. 4, but with a unique and novel balloon design with particular advantages as discussed herein. A first end 420 of the balloon 406 may extend from a distal end 408 of a catheter 402. In the depicted embodiment, the balloon 406 is tube-shaped. A proximal end of the balloon may be in fluid communication with a lumen 404 of the catheter, which may be used for inflating the balloon 406. As the balloon extends from its first end 420 to its second end 422, the balloon 406 includes a helical portion 462 that wraps around the catheter 402 in a spiral or coil pattern as it extends proximally. In other words, the second end 422 of the balloon is located proximally relative to the balloon's first end 420 (using the catheter 402 as the frame of reference).

Optionally, the balloon 406 includes a post 464 at its first end 420, which is shown in FIGS. 9-11. Notably, the “post 464” may be a portion of the balloon itself, such as a relatively small-diameter section of the balloon 406 that extends in the longitudinal direction of the catheter 402, for example. The helical portion 462 of the balloon coils not only around the distal end 408 of the catheter 402, but also around the post 464 of the balloon 406. Advantageously, the post 464 may increase the overall outer diameter of the balloon 406 relative to embodiments without a post 464 while still providing a compact design for uterus insertion when the balloon 406 is not inflated.

The balloon 406 may be affixed at points of contact with other balloon surfaces and/or the catheter 402. For example, the helical portion 462 of the balloon 406 may be secured to the post 464 at securement points 466 such that the coiled relationship is maintained during deployment of the device, even prior to balloon inflation.

The catheter 402 may have multiple lumens, such as an aspiration lumen 405 and an inflation lumen 404. These lumens may be the same size, or not. For example, the aspiration lumen 405, which may be used for aspiration, the application/delivery of a fluid, for drainage, etc., may be larger than the inflation lumen 404. The inflation lumen 404 is in fluid communication with an interior of the balloon, and therefore the balloon may be inflated through an inflation inlet 427 located at a proximal end 414 of the tamponade device 400. As shown in FIG. 11, an aspiration port 418 may be included for allowing flow to and from the aspiration lumen 405 (for purposes of aspiration, drainage, etc.). In some embodiments (including the depicted embodiment), more than one aspiration port 418 may be included, and their locations may be anywhere suitable along the length of the catheter 402. For example, the aspiration ports 418 may be included generally beneath the helical portion 462 of the balloon 406 on a side of the catheter 402 facing away from the post 464. Advantageously, ports 418 in this location may allow for flow beneath the helical portion 462 of the balloon 406, causing a current of fluid either through the helical/spiral cavity/channel extending adjacent to the catheter 402, through openings between adjacent helical sections of the balloon 406, or both.

The balloon 406 may have several advantageous features relative to existing balloons. For example, the balloon 406 has a relatively low profile but also a high degree of column-strength relative to certain existing balloons. Further, control over the inflated size is ideal when adapted for use in the uterus since overinflated/underinflated results are closer to the ideal result relative to typical cylindrical balloons. The helical pattern also creates passageways for drainage.

The above described system and method may be used to provide internal compression to the uterus in addition to other systems and methods that provide external compression to the uterus.

The balloon 406 may be formed with any suitable construction and/or method. For example, the balloon 406 may be formed via dip molding or injection molding (e.g., with liquid silicone rubber (LSR). In another contemplated method, the balloon 406 may be constructed of a sheet of material that has been cut to shape, then two or more pieces of this sheet may be joined at the edges to create a pocket that takes on a 3D shape when inflated. Alternatively, the balloon 406 may be constructed of tubes of material that are heated and inflated into a cavity that causes the resulting balloon to re-form and take on the desired shape. In yet another alternative, the balloon 406 may be constructed of tubes of material that are inflated and then formed into the desired shape and restrained through bonding so that they take on the desired shape when inflated. Any other suitable construction/manufacturing method may additionally or alternatively be used.

While the present invention has been described in terms of certain preferred embodiments, it will be understood that the invention is not limited to these disclosed embodiments as those having skill in the art may make various modifications without departing from the scope of the following claims.

Having described various aspects of the subject matter above, additional disclosure is provided below that may be consistent with the claims originally filed with this application.

In aspect 1, a tamponade device may include one or more of the following: a catheter, where a catheter lumen extends from a proximal end of the catheter to a distal end of the catheter; an inflatable balloon located at the distal end of the catheter; and a constraint at least partially surrounding the inflatable balloon, and where in an expanded configuration, the constraint contacts an outer perimeter surface of the balloon such that the constraint limits a radial expansion of the balloon when the balloon is inflated.

In aspect 2, the tamponade device of aspect 1 includes the constraint having a net structure with a plurality of segments, where the plurality of segments are arranged to defined a plurality of openings, and where at least a portion of the balloon extends radially outward through the openings when the balloon is inflated.

In aspect 3, the net structure from aspect 2 includes an angled profile portion and a central portion, the central portion being generally cylindrical when the net structure is in the expanded configuration.

In aspect 4, at least one of a proximal end and a distal end of the net structure from either of aspects 2-3 includes a section of a tube made of a polymer material, and where the segments of the net structure include the polymer material.

In aspect 5, the net structure from any of aspects 1-4 is secured to at least one tube having a lumen extending therethrough, and where the at least one tube is in fluid communication with an inlet at a proximal end of the tamponade device.

In aspect 6, the tube from aspect 5 includes at least one opening at a distal end of the tube for delivering a fluid flowing through the lumen of the tube.

In aspect 7, the tube from any of aspects 5-6 is woven with segments of a central portion of a net of the constraint.

In aspect 8, the tamponade device of any of aspects 1-7 includes at least one of a distal end of the constraint and a proximal end of the constraint that is slidable along the catheter when the tamponade device transitions from a delivery configuration to the expanded configuration.

In aspect 9, the distal end and the proximal end of the constraint from aspect 8 are closer together in the expanded configuration relative to the delivery configuration.

In aspect 10, the lumen from any of aspects 1-9 is configurated as an inflation lumen configured to deliver a fluid to the balloon during balloon inflation, where the catheter also includes a second lumen configured for drainage of a body fluid.

In aspect 11, a tamponade device may include one or more of the following: an inflatable balloon located at a distal end of the tamponade device; and at least one tube contacting an outer surface of the balloon when the balloon is inflated, where the tube includes a plurality of openings providing a flow path between a lumen extending through the tube and an area outside the lumen.

In aspect 12, a constraint of the tamponade device of aspect 11 may at least partially surround the inflatable balloon, where in an expanded configuration, the constraint contacts an outer perimeter surface of the balloon such that the constraint limits a radial expansion of the balloon when the balloon is inflated.

In aspect 13, the tamponade device of any of aspects 11-12 may include the tube at least partially forming a net of the constraint.

In aspect 14, the net from aspect 13 includes a plurality of net openings.

In aspect 15, at least a portion of the balloon from any of aspects 11-14 extends radially outward through the openings when the balloon is inflated.

In aspect 16, the tube from any of aspects 11-15 extends along the balloon in a helical pattern, where at least one polymer strip extends in an opposite pattern to form a criss-crossing pattern overlaying the outer surface of the balloon.

In aspect 17, a tamponade device may include one or more of the following: a catheter, where a catheter lumen extends from a proximal end of the catheter to a distal end of the catheter; and an inflatable balloon located at the distal end of the catheter, where the inflatable balloon includes a first end secured to the distal end of the catheter and a second end opposite the first end, and where the inflatable balloon includes a helical portion between the first end and the second end that coils around at least a portion of the catheter.

In aspect 18, the first end of the inflatable balloon from aspect 17 includes a post that extends along the distal end of the catheter, where the helical portion of the inflatable balloon coils around at least a portion of the post.

In aspect 19, the catheter from any of aspects 17-18 includes at least one port located beneath the helical portion of the inflatable balloon, the at least one port leading to a first lumen.

In aspect 20, the catheter from any of aspects 17-19 includes the first lumen and a second lumen, and where the second lumen is in fluid communication with the inflatable balloon such that the inflatable balloon is inflatable via the second lumen.

1. A tamponade device comprising:
a catheter comprising a proximal end, a distal end, and a catheter lumen, wherein the catheter lumen extends between the proximal end of the catheter and the distal end of the catheter;
an inflatable balloon located at the distal end of the catheter; and
a constraint at least partially surrounding the inflatable balloon,
wherein in an expanded configuration, the constraint contacts an outer perimeter surface of the inflatable balloon such that the constraint limits a radial expansion of the inflatable balloon when the inflatable balloon is inflated.

2. The tamponade device of claim 1,
wherein the constraint comprises a net structure having a plurality of segments,
wherein the plurality of segments are arranged to defined a plurality of openings, and
wherein at least a portion of the inflatable balloon extends radially outward through the plurality of openings when the inflatable balloon is inflated.

3. The tamponade device of claim 2, wherein the net structure comprises an angled profile portion and a central portion, the central portion being generally cylindrical when the net structure is in the expanded configuration.

4. The tamponade device of claim 2, wherein at least one of a proximal end or a distal end of the net structure comprises a section of a tube made of a polymer material, and wherein the plurality of segments of the net structure comprise the polymer material.

5. The tamponade device of claim 2, wherein the net structure is secured to at least one tube having a lumen extending therethrough, and wherein the at least one tube is in fluid communication with an inlet at a proximal end of the tamponade device.

6. The tamponade device of claim 5, wherein the at least one tube comprises at least one opening at a distal end of the at least one tube for delivering a fluid flowing through the lumen of the at least one tube.

7. The tamponade device of claim 5, wherein the at least one tube is woven with one or more of the plurality of segments of a central portion of the net structure of the constraint.

8. The tamponade device of claim 1, wherein at least one of a distal end of the constraint or a proximal end of the constraint is slidable along the catheter when the tamponade device transitions from a delivery configuration to the expanded configuration.

9. The tamponade device of claim 8, wherein the distal end and the proximal end of the constraint are closer together in the expanded configuration relative to the delivery configuration.

10. The tamponade device of claim 1, wherein the catheter lumen is configurated as an inflation lumen configured to deliver a fluid to the inflatable balloon during balloon inflation, and wherein the catheter comprises a second lumen configured for drainage of a body fluid.

11. A tamponade device comprising:
an inflatable balloon located at a distal end of the tamponade device; and
at least one tube contacting an outer surface of the inflatable balloon when the inflatable balloon is inflated,
wherein the at least one tube comprises a plurality of openings providing a flow path between a lumen extending through the at least one tube and an area outside the lumen.

12. The tamponade device of claim 11, comprising a constraint at least partially surrounding the inflatable balloon, wherein in an expanded configuration, the constraint contacts the outer surface of the inflatable balloon such that the constraint limits a radial expansion of the inflatable balloon when the inflatable balloon is inflated.

13. The tamponade device of claim 12, wherein the at least one tube at least partially forms a net of the constraint.

14. The tamponade device of claim 13, wherein the net comprises a plurality of net openings.

15. The tamponade device of claim 14, wherein at least a portion of the inflatable balloon extends radially outward through one or more of the plurality of net openings when the inflatable balloon is inflated.

16. The tamponade device of claim 12, wherein the tube extends along the inflatable balloon in a helical pattern, and wherein at least one polymer strip extends in an opposite pattern to form a criss-crossing pattern overlaying the outer surface of the inflatable balloon.

17. A tamponade device comprising:
a catheter comprising a proximal end, a distal end, and a catheter lumen, wherein the catheter lumen extends between the proximal end of the catheter and the distal end of the catheter; and
an inflatable balloon located at the distal end of the catheter,
wherein the inflatable balloon comprises a first end secured to the distal end of the catheter and a second end opposite the first end, and
wherein the inflatable balloon comprises a helical portion between the first end and the second end that coils around at least a portion of the catheter.

18. The tamponade device of claim 17, wherein the first end of the inflatable balloon comprises a post that extends along the distal end of the catheter, and wherein the helical portion of the inflatable balloon coils around at least a portion of the post.

19. The tamponade device of claim 17, wherein the catheter comprises a second lumen and at least one port located beneath the helical portion of the inflatable balloon, the at least one port leading to the second lumen.

20. The tamponade device of claim 19, wherein the catheter lumen is in fluid communication with the inflatable balloon such that the inflatable balloon is inflatable via the catheter lumen.
